(19) United States
(12) Patent Application Publication
(10) Pub. No.: US 2019/0255639 A1
(43) Pub. Date: Aug. 22, 2019

(54) NIOBIUM COATED SLEEVES FOR JOINING NICKEL TITANIUM SHAPE MEMORY COMPONENTS FOR MEDICAL DEVICES

(71) Applicant: Medplate LifeSciences Corporation, Bridgeville, PA (US)

(72) Inventors: David S. Grummon, East Lansing, MI (US); Richard T. Briganti, Bala Cynwyd, PA (US)

(73) Assignee: Medplate LifeSciences Corporation, Bridgeville, PA (US)

(21) Appl. No.: 16/271,593

(22) Filed: Feb. 8, 2019

Related U.S. Application Data
(60) Provisional application No. 62/631,867, filed on Feb. 18, 2018.
(60) Provisional application No. 62/791,693, filed on Jan. 11, 2019.

(51) Int. Cl. B23K 1/00; B21F 45/00; A61L 31/02

(52) U.S. Cl. CPC B23K 1/0008; B23K 2101/06; A61L 31/022; B21F 45/008

(57) ABSTRACT

A method of joining two metal components for forming medical devices. The method includes placing a first and second metal component into a sleeve, the first sleeve composed of a nickel titanium alloy and having niobium deposited thereon, and heating the first sleeve so the niobium melts and reacts to form a joint joining the first and second components. A medical device is also provided having a first region having a first property, a second region having a second property different than the first property and a joint formed by a niobium coated nickel titanium alloy sleeve melted onto a first section of the first region and a second section of the second region.

### BACKGROUND OF THE INVENTION

This application claims priority from provisional application 62/631,867, filed Feb. 18, 2018, and provisional application Ser. No. 62/791,693, filed Jan. 11, 2019. The entire contents of each of these applications are incorporated herein by reference.

### BACKGROUND OF THE INVENTION

### Field of the Invention

This application relates to metallurgical joining of components of shape memory and superelastic nickel titanium alloys to other components of shape memory and superelastic nickel titanium alloys, and/or to other biocompatible metals or metal alloys, by reactive eutectic brazing to form medical and other devices.

### Background

U.S. Pat. No. 7,896,222 (the “222 patent”) discloses reactive eutectic brazing for joining of shape memory and superelastic nickel titanium alloys to each other or other metals or metal alloys. As disclosed in the '222 patent, niobium reacts with Nitinol shape memory alloys to produce a eutectic liquid that may be used to form a strong metallurgical joint. Thus, pure niobium can be used as a braze-foil by way of its well-understood contact melting reaction with NiTi (nitinol) alloys. Niobium is brought into contact with a NiTi alloy and heated to 1170 degrees C., and then quenched, in order to effect the braze. Pure niobium alloyed with another metal capable of forming an alloy with niobium is also disclosed.

However, how and where to apply the required niobium to form the joint requires great care as the niobium does not melt by itself. It needs to be in direct contact with NiTi for melting to occur, and a very significant amount of NiTi enters the eutectic melt as well. When joining NiTi alloys, it is undesirable to bring niobium in direct contact with the NiTi objects to be joined, because those same objects will be significantly attacked as the contact melting reaction proceeds. When pure niobium reacts with a NiTi alloy, approximately two volumes of NiTi metal will enter the eutectic liquid for each volume of niobium in the reaction. The amount of niobium also requires great care as too much niobium can damage the components to be joined and too little niobium could result in an insufficiently strong joint causing fracture of the device. Therefore, a balance needs to be achieved between these opposing factors.

Thus, if the location, form, and amount of added niobium metal are not carefully controlled, deleterious attack of the structures-to-be-joined can occur. Also, the capillary flow of the eutectic liquid must be managed properly, so that eutectic liquid metal flows to regions of the joint structure where it is needed for formation of the joint. Therefore, the need exists for connecting such components without damaging the components to be joined while still providing a sufficiently strong joint connecting the components.

Additionally in forming small devices such as medical devices, the processes for applying niobium to the small components in the desired quantity and correct locations for formation of a strong metallurgical joint (by reactive brazing) can be difficult and expensive. Therefore, it would be advantageous to provide a process to simplify and reduce the cost of manufacturing such devices using the reactive brazing process of the '222 patent, while ensuring a sufficiently strong and effective joint is formed.

### SUMMARY

This present invention provides metallurgical joining of shape memory and superelastic nickel titanium alloys to each other, and/or to other metals or metal alloys (e.g., a biocompatible metal or metal alloy) by reactive eutectic brazing, thereby forming a strong metallurgical joint.

In accordance with one aspect of the present invention, two components are joined end to end by a niobium-coated NiTi alloy sleeve. In accordance with another aspect of the present invention, a niobium-coated sleeve is joined to a single inner component. In accordance with yet another aspect of the present invention, the niobium coated sleeve forms a connector for two axially spaced longitudinally aligned components. In accordance with another aspect of the present invention, multiple components are joined by multiple niobium sleeves forming multiple joints along the device. Each of these variations is discussed in detail below.

In some embodiments, a cylindrical niobium-coated NiTi-alloy sleeve is utilized to join and reinforce two components such as long thin tubes made from dissimilar biomedical alloys. Differently configured sleeves and different geometries are discussed herein. The components joined can be solid (only having a single diameter) or alternatively hollow (having an OD (outer diameter) and an ID (inner diameter)).

Three different process protocols are disclosed herein. The first process specifies a set of guidelines for a niobium metal coating to be applied, in a batch process, to a metal feed stock. The second process describes a laser-micromachining procedure for fenestrating, slotting, and dicing this feed stock (after coating with niobium or in some embodiments prior to coating with niobium) into many tiny sleeves, preferably cylindrical, these later being used to create and reinforce butt-joints in hypodermic tubing segments (or other components, e.g., wires) of dissimilar biomedical alloys. The third process describes a galvo-controlled laser based vacuum brazing process, based on the eutectic brazing U.S. Pat. No. 7,896,222, however, here it utilizes the niobium coated cylindrical sleeve to effect a metallurgical, robust, and hermetic joint between dissimilar tubes or wires.

In accordance with one aspect of the present invention, a method of joining two metal components is provided comprising a) positioning a first metal component in a first end of a sleeve, the sleeve composed of a nickel titanium alloy and having niobium deposited thereon; b) positioning a second metal component in a second end of the sleeve; and c) increasing the temperature of the sleeve so the niobium reacts and melts to form a joint joining the first and second components.

In some embodiments, the first and second components are laser heated and heat transfers to the sleeve to increase the temperature.

In some embodiments, the first component is composed of one of platinum, tantalum or stainless steel, e.g., annealed 316 or 304 stainless steel, or stainless steel plated or clad coated with NiTi, Pt or Ta (which expands the temperature processing window) and the second component is composed of a shape memory or superelastic nickel titanium alloy. In some embodiments, the first component is composed of a shape memory or superelastic nickel titanium alloy.

In some embodiments, the sleeve is composed of a shape memory or superelastic nickel titanium alloy.

In some embodiments, the first component has a flexibility less than a flexibility of the second component at room temperature. In some embodiments, the first component has a flexibility less than a flexibility the second component at body temperature. The first and second components can have other varying properties.

In some embodiments, the first and second components are placed with ends in abutment within the sleeve prior to melting. The sleeve in preferred embodiments, avoids direct contact of the niobium and the first and second components underlying the sleeve.

In some embodiments, the sleeve has a plurality of fenestrations (openings) in a wall of the sleeve for flow of the melted niobium into contact with a surface of the first and second components underlying the sleeve. Various placements/arrangements and number of the fenestrations are disclosed. In some embodiments, the plurality of fenestrations are spaced from edges of the sleeve and spaced from the center point of the sleeve.

In some embodiments, the sleeve has a slot at the first and second ends for flow of eutectic liquid into a gap between the inner diameter of the sleeve and the outer diameter of the first and second components.

In accordance with another aspect of the present invention, a method of forming a joint between a first component composed of nickel titanium alloy and a second component composed of a biocompatible metal or metal alloy is provided, the method comprising placing a niobium coated sleeve over a region of the first and second components and reactively brazing the sleeve to the first and second components to form a brazed joint between the first and second components.

In some embodiments, the method further includes the step of placing the first and second components within opposing ends of the sleeve and in end to end abutment prior to reactive brazing. In some embodiments, during reactive brazing, the niobium flows around edges of the sleeve and into a gap between the inner surface of the sleeve and outer surface of the components. In some embodiments, during reactive brazing, the niobium flows through openings in the sleeve, the openings communicating with an outer surface of the first and second components.

In some embodiments, the biocompatible metal is a nickel titanium alloy; in other embodiments, the biocompatible metal is one of platinum, titanium or stainless steel coated or plated with another metal or alloy of the foregoing. In some embodiments, the first and second components are superelastic and/or shape memory.

In some embodiments, a ratio of niobium coating thickness to a sleeve wall thickness is ≤½; in other embodiments it is less than ¼; and in other embodiments the niobium coating thickness is between about 1% and about 15% of the sleeve wall thickness. In some embodiments, the niobium coating thickness on the sleeve is between one half the sleeve wall thickness at maximum and one half the thickness of the sleeve to inner component gap.

In accordance with another aspect of the present invention, a method of forming niobium coated nickel titanium alloy sleeves for use for joining a first component of shape memory or superelastic material to a second component of a biocompatible metal is provided, the method comprising:
a) providing a bulk stock of tubes of nickel titanium alloy;
b) depositing niobium on an outer surface of the tubes;
c) either before or after step (b), laser cutting openings or slots into an outer wall of the tubes; and
d) laser cutting the stock into individual tubes to form outer sleeves for joining the first and second components by reactive eutectic brazing.

In some embodiments, the individual tubes have a first opening at a first end to receive the first component and a second opening at a second end to receive the second component so the tubes overlie a region of the first and second components.

In accordance with another aspect of the present invention, a medical device is provided having a first region having a first property, a second region having a second property different than the first property and a joint formed by a niobium coated nickel titanium alloy sleeve melted onto a first section of the first region and a second section of the second region.

In some embodiments, the device includes a third region of a third property different than the first property and the second property, and a second joint is formed by a second niobium coated nickel titanium sleeve melted onto a third section of the second region and a fourth section of the third region.

In some embodiments, the first property is a first stiffness (Young's modulus) and the second property is a second stiffness greater than the first stiffness. In other embodiments, the first property is a first yield stress and the second property is second yield stress greater than the first yield stress. In some embodiments, the first region is distal of the second region; in other embodiments, the second region is distal of the first region. With shape memory, the stiffness changes with temperature.

In accordance with another aspect of the present invention, a medical device is provided having a first component having a first property, a second component having a second property different than the first property and a nickel titanium sleeve bridging the first and second component. The device has a first joint formed by the nickel titanium alloy sleeve having niobium thereon at a first end melted onto a first section of the first component and a second joint formed by the nickel titanium alloy sleeve having niobium thereon at a second end melted onto a second section of the second component.

In some embodiments, the sleeve has a flexibility less than a flexibility of the first component.

In some embodiments, the first component is a metal braided structure.

In accordance with another aspect of the present invention, a medical device is provided having a first region, a second region and a third region, wherein the Af temperature of each of the regions is different, and at least the first region is composed of a nickel titanium alloy and the first and second regions are formed by different components, the first and second components each containing niobium thereon.

In accordance with another aspect of the present invention, a product by a process is provided comprising a medical device formed by a laser brazing process, the device formed by first and second components joined together by a nickel-titanium alloy sleeve having niobium thereon and laser brazed to react and melt to flow to the first and second components extending into the sleeve thereby forming a joint to join the first and second components.

In some embodiments, the sleeve avoids direct contact with the niobium and the first and second components extending into the sleeve. Preferably, the niobium for reactive brazing is not applied to the first and second components extending into the sleeve. The sleeve can be coated by various processes such as by a PVD process of sputtering.

In some embodiments, the ratio of niobium coating thickness to a sleeve wall thickness is ≤½ and the niobium coating thickness on the sleeve is between one half the sleeve wall thickness at maximum and one half the thickness of the sleeve to inner component gap.

### BRIEF DESCRIPTION OF THE DRAWINGS

So that those having ordinary skill in the art to which the subject invention appertains will more readily understand how to make and use the surgical apparatus disclosed herein, preferred embodiments thereof will be described in detail hereinbelow with reference to the drawings, wherein:

FIG. 1A is a perspective view of a batch (bulk set) of tubes for forming sleeves of the present invention;

FIG. 1B is a perspective view of a batch of tubes for forming sleeves of the present invention, the tubes laser cut to form openings in the wall of the tubes;

FIG. 2 is a side view of one embodiment of the sleeve of the present invention positioned over two components (tubes) to be joined;

FIG. 3A is a perspective view of an embodiment of the sleeve of the present invention having micro fenestrations;

FIG. 3B is a perspective view of an alternate embodiment of the sleeve of the present invention having micro fenestrations, and further showing the sleeve positioned over two components to be joined;

FIG. 4 is a perspective view of another embodiment of the sleeve of the present invention having slotted ends;

FIG. 5 is a perspective view of another embodiment of the sleeve of the present invention having slotted ends;

FIG. 6A is a cross-sectional view of another embodiment of the sleeve of the present invention having slotted swaged ends;

FIG. 6B is a perspective view illustrating the sleeve of FIG. 6A prior to swaging one of the slotted ends;

FIGS. 7A and 7B illustrate the manufacturing steps to form the slotted swaged sleeve of FIG. 6A wherein FIG. 7A illustrates the swage tools engaging the end of the sleeves on the batch and FIG. 7B illustrates the resulting swaged regions of the sleeve prior to separation from the batch;

FIGS. 7C, 7D and 7E illustrate an alternate manufacturing process to form the swaged ends wherein FIG. 7C illustrates the forming disks, FIG. 7D is a front view of the disks and FIG. 7E is a cross-sectional view taken along line A-A of FIG. 7D;

FIG. 8 is a cross-sectional view showing the sleeve of FIG. 6A placed over two tubes to be joined;

FIG. 9 is a perspective view of another embodiment of a slotted sleeve forming a collar for attachment over an inner tube;

FIG. 10 is a perspective view of an alternate embodiment of the sleeve of the present invention having a having a C-shaped cross-section;

FIG. 11 is a side view showing an alternate embodiment of the sleeve of the present invention positioned over two axially spaced tubes to be joined;

FIG. 12 is a top view of a niobium coated planar member of an alternate embodiment of the present invention for joining two planar components;

FIG. 13 is a side view showing an alternate embodiment of the sleeve of the present invention for joining two axially spaced components;

FIG. 14 is a side view showing an alternate embodiment of the sleeve of the present invention in the form of a niobium coated braid for joining two axially spaced components;

FIG. 15 is a side view of an alternate embodiment of the sleeve of the present invention in the form of a niobium coated braid joined to a single component;

FIG. 16A is a perspective view of an alternate embodiment of the sleeve of the present invention for joining two components;

FIG. 16B is a side view of the sleeve of FIG. 16A;

FIG. 16C is a transverse cross-sectional view taken along line A-A of FIG. 16B;

FIG. 17A is a perspective view of an alternate embodiment of the sleeve of the present invention for joining two components;

FIG. 17B is a side view of the sleeve of FIG. 17A;

FIG. 17C is a transverse cross-sectional view taken along line A-A of FIG. 17B;

FIG. 18A is a perspective view of the sleeve of FIG. 17A with two components to be joined inserted into the sleeve;

FIGS. 18B and 18C are side views of the sleeve of FIG. 18A with two components to be joined inserted into the sleeve;

FIG. 18D is a longitudinal cross-sectional view of the sleeve and two components of FIG. 18A;

FIG. 19A is a chart depicting one example of varying temperatures of the device sections in accordance with one embodiment of the present invention;

FIG. 19B is a chart depicting two examples of a device of three varying sections in accordance with embodiments of the present invention;

FIG. 19C is a chart depicting an example of a device of five varying sections in accordance with embodiments of the present invention;

FIG. 20 is a schematic view of a system of the present invention for changing the stiffness of the device; and

FIG. 21 illustrates the electrical contacts on the device for use with the system of FIG. 20.

### DESCRIPTION OF PREFERRED EMBODIMENTS

As disclosed in U.S. Pat. No. 7,896,222, niobium reacts with Nitinol shape memory alloys to produce a eutectic liquid that may be used to form a strong metallurgical joint. Thus, pure niobium can be used as a braze-foil by way of its well-understood contact melting reaction with NiTi (nitinol) alloys. Niobium is brought into contact with a NiTi alloy and heated to 1170 degrees C., and then quenched, in order to effect the braze.

How and where to apply the required niobium requires great care. Unlike a solder, or a conventional braze foil, the niobium does not melt by itself. It needs to be in direct contact with NiTi for melting to occur, and a very significant amount of NiTi enters the eutectic melt as well. The eutectic liquid composition is fixed and is thought to be approximately 76 atom % (NiTi) and 24 atom % niobium and forms at 1170 C. For this reason, when joining NiTi alloys, it is undesirable to bring niobium in direct contact with the NiTi objects to be joined, because those same objects will be significantly attacked as the contact melting reaction proceeds.

Since direct contact is undesirable, the present invention provides a process to avoid such direct contact. The process brings niobium in contact with NiTi in a sacrificial structure that can contribute the (NiTi) atoms to the melt pool, but without damaging the underlying components to be joined. This is possible because once formed, the eutectic liquid flows hydraulically with great ease. Since this liquid is more than one third titanium atoms, which are very reactive, it readily dissolves surface oxides and wets completely virtually any metal surface. It flows with ease into capillary crevices and can thus fill them, bonding surfaces metallurgically, and doing so without the use of fluxes.

The present invention enables making strong metallurgical joints between extremely fine solid or hollow tubes, or other members. For these joints, only very small quantities of niobium are needed and may be supplied by any of a number of deposition or coating processes. The use of organic binders is precluded by the need to exclude contamination. Furthermore, such joints require some degree of reinforcement in order to be mechanically robust. The present invention uses a small cylindrical sleeve covering the joint region between the inner components, e.g., small tubes (hypotubes) or solid tubes or wires, to be joined, the sleeve coated with a controlled amount of niobium, and reactively brazed to the inner components as in the brazing technique disclosed in U.S. Pat. No. 7,896,222.

If the niobium for reactive brazing is deposited onto the inner tubes-to-be-joined before they are inserted into the sleeve, contact melting occurs between niobium and the tubes-to-be-joined, potentially weakening them.

Furthermore, in dealing with very small physical elements such as hypotubes, it could be difficult and expensive to apply such niobium, in the correct quantity, and at the correct location, to each of the individual objects (components) to be joined. That is, techniques for efficiently applying a niobium coating or film to small components such as by physical vapor deposition (PVD), are not easily performed. The present invention provides a simpler way to utilize NiTi and niobium to effect a joint between two components composed of nickel titanium alloy (nitinol—NiₓTi_{y}), or between two NiTi components having different compositions, or between a NiTi alloy and another metal or metal alloy (e.g., biocompatible metal or metal alloy for medical applications). The processes can facilitate manufacture and reduce manufacturing costs.

The present invention has application to construct surgical tools or other biomedical devices (or other non-medical devices), in which the special mechanical properties of superelastic nitinol (NiTi) alloys (SE NiTi) are combined with also-desired properties of shape-memory nitinol (SM NiTi) formulations, or of ductile stainless steel alloys or other metal alloys or metals. A biocompatible brazed joint can be made between NiTi alloy components if a small amount of pure niobium metal, in the joint region, enters into a contact-eutectic-melting reaction with the NiTi alloys in the joint. This invariant reaction produces a liquid with eutectic composition that is rich in titanium such that it flows readily on, and along, surfaces and into capillary spaces. When pure niobium reacts with a NiTi alloy, approximately two volumes of NiTi metal will enter the eutectic liquid for each volume of niobium in the reaction. Thus, if the location, form, and amount of added niobium metal are not carefully controlled, deleterious attack of the structures-to-be-joined can occur. Also, the capillary flow of the eutectic liquid must be managed properly, so that eutectic liquid metal flows to regions of the joint structure where it is needed for formation of the joint.

In the present invention, a carefully-controlled volume of pure niobium metal is applied or deposited (by any one of many possible physical deposition methods) to the outside diameter (outer surface) of a metal component, e.g., a wire or tube such as a thin-walled tube, that is designed to be used as a coupling sleeve for a brazed joint with another component or between two components such as wires or tubes, e.g., two hypodermic-gauge tubes, of dissimilar biomedical alloys. The sleeve can be composed of nitinol (nickel titanium alloy) material such as NDC's SE-508 material. The present invention utilizes an economical industrial method for batch niobium-coating of NiTi tubes to make a precursor material, and can also utilize laser micromachining of the individual sleeve-tubes to enhance their functionality during the joint assembly process, and during the subsequent thermal brazing process. The process enables joining for example components of the following material, however, it should be appreciated that the present invention is not limited to such materials:
i. SE NiTi-to-SE NiTi
ii. SE NiTi-to-SM NiTi
iii. SM NiTi-to-SM NiTi
iv. SE NiTi-to-Pt
v. SM NiTi-to-Pt
vi. SE NiTi-to-Ta
vii. SM NiTi-to-Ta

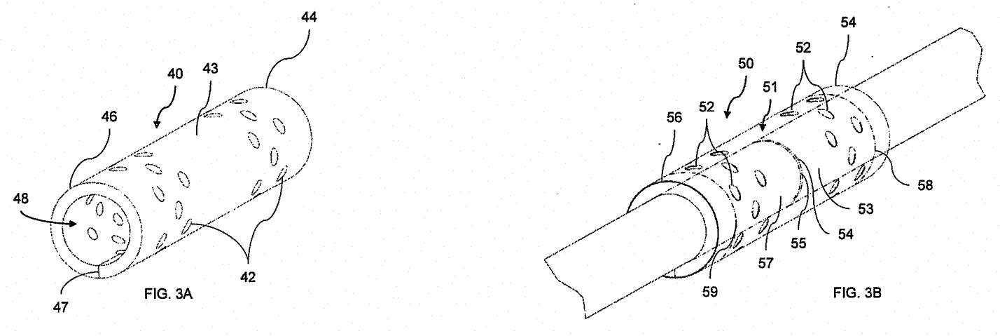

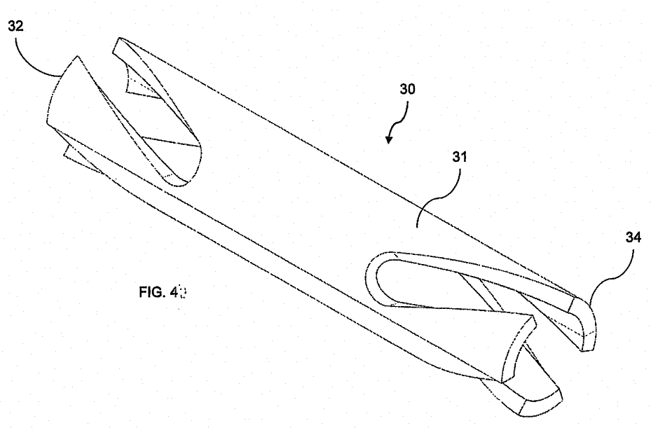

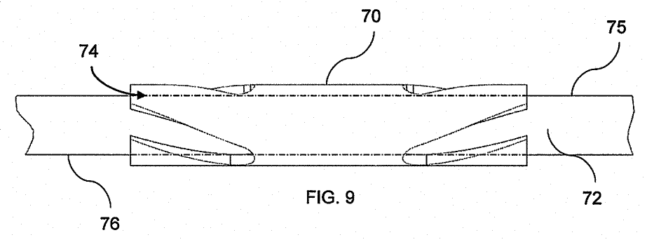

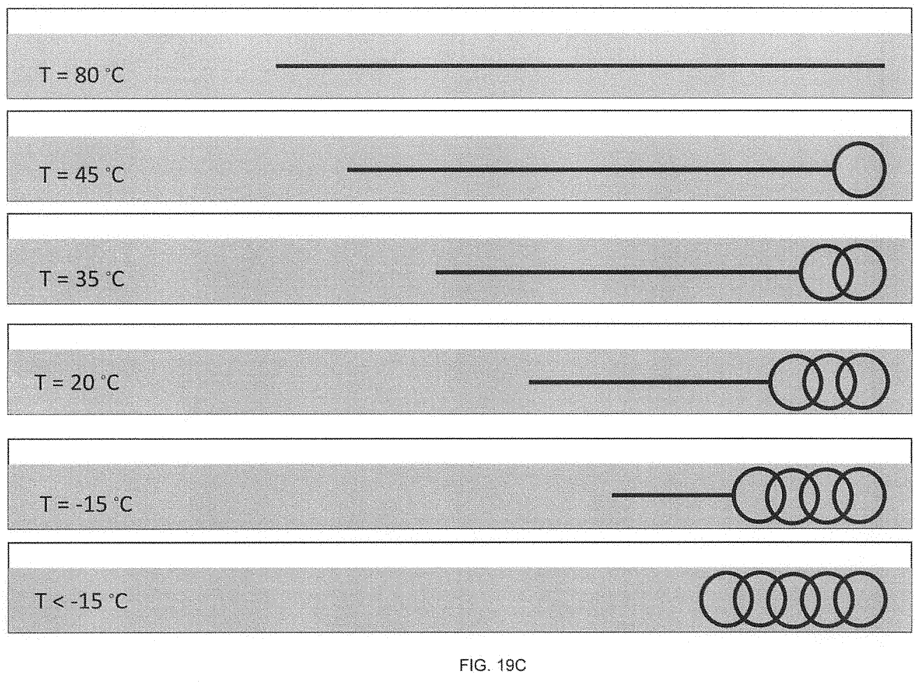

The inner component has an end which is inserted into an opening in the outer component (the sleeve) to ultimately form a device having different properties along its length. In some embodiments by way of example, the device could be a guidewire or a hypotube formed to have different properties along its length. Thus, the first component can form a proximal section of the device and the second component can form a distal section of the device, and the ratio of the length of the inner component to the outer component can vary dependent on the desired length for the more rigid section of the device. Depending on the selected lengths, one of the components could also form an intermediate section of the device. For example, the second component can be composed of a material of less rigidity so that it can form a more flexible distal end of the device. In alternative embodiments, another component is inserted into another opening in the outer component (sleeve) at the end opposite the end the first component was inserted to form another device section. For guidewires, it is desirable to have the distal section the most flexible (less stiff) or the distal section more malleable or more formable than the proximal section. In other devices, it might be desirable to have the proximal section or an intermediate section most flexible (less stiff). It can be appreciated that by joining these materials using the processes of the present invention disclosed herein, varying properties can be provided along a length of the device. These include by way of example, varying stiffnesses, varying transition temperatures, varying dimensions (e.g., tapers), varying formability or malleability, varying radiopacity, varying structure such as tubes joined to braids, etc. Additionally, multiple joints can be provided so that the device can be formed of multiple components, with the niobium sleeve utilized to join the two adjacent components. Thus, it should be appreciated that one component can be joined to the sleeve, (e.g., FIGS. 9 and 15), two components can be joined to the sleeve (e.g., FIGS. 2 and 3B) as well as multiple components with multiple joints (utilizing multiple niobium sleeves) can be joined to form the device. That is, the device can have multiple joints (each utilizing the sleeve described herein), with each joint joining two components (e.g., represented by the chart of FIG. 19A). These are discussed in more detail below.

Also, in alternate embodiments the sleeve can be bifurcated to have more than two open ends, e.g., have a Y-shape to receive three components, e.g., a component extending into each leg of the “Y”. Additionally, it is also contemplated that multiple components can be inserted into an end of the sleeve. For example, the “first component” and/or the “second component” can be a single component or multiple components inserted into an end of the sleeve. If multiple components, the components can be D-shaped (less than 360 degrees when viewed in transverse section) and preferably together form a full 360 degrees extending into the end of the sleeve. If not forming the 360 degrees, the sleeve shape preferably conforms to the shape (<360 degrees) of the combined inner components inserted in the end of the sleeve.

The present invention utilizes an economical industrial method for batch niobium-coating of NiTi tubes to make a precursor material, and can also utilize laser micromachining of the individual tubes (sleeves) to enhance their functionality during the joint assembly process, and during the subsequent thermal brazing process. In preferred embodiments, a continuous wave rather than a pulse wave is utilized for laser brazing. Also, in preferred embodiments, XYZ 3-Axis simultaneous scanning method is utilized rather than a fixed laser. Furnace brazing can be used to join very thick workpieces (thick samples require more power to heat) or in a batch process where multiple joints are brazed at one time (instead of laser brazing where typically one joint is created at a time). One advantage of laser brazing is that unlike furnace brazing, the entire device is not exposed to the brazing temperatures but just the joints. In some embodiments by way of example, laser brazing can be used for heating a solid wire of 0.040 inch diameter.

In one embodiment, the sleeve (outer component) is coated with niobium by a PVD process such as sputtering. Other deposition methods/techniques for applying niobium so it sticks to the outer surface of the sleeve, thereby providing a niobium coating or film (having a coating thickness), are also contemplated. These include by way of example pulsed laser deposition, vacuum evaporation, laser powder consolidation (as in 3D printing), plasma spray, thermal spray, kinetic spray (spraying fine local powders), laser cladding, etc. Tube co-extrusion could also be utilized. Preferably, the deposition process is applied to precursor nitinol stocks, such as nitinol sheets, wires, strips, tubes, etc., which are subsequently cut into the sleeves, or other joint-enabling structures, for joining two components, e.g., two tubes. After the deposition process, the nitinol stock is preferably laser cut to form the individual sleeves. During the individual sleeve cutting process, the laser can be also be utilized to cut other features into the sleeves such as fenestrations and/or slots, as discussed below. In some embodiments, the niobium is applied to the nitinol stock then the features (e.g., fenestrations or slots described below) are laser cut followed by laser cutting the stock into individual sleeves; in other embodiments, the features (e.g., fenestrations or slots) are laser cut in the nitinol stock then the niobium is applied to the stock followed by laser cutting the stock into individual sleeves; in other less preferred embodiments, the features (e.g., fenestrations or slots) are laser cut in the nitinol stock then the stock is laser cut into individual sleeves followed by applying niobium to individual sleeves. Other sequences of these steps are also contemplated.

The sleeve (also referred to herein as “the niobium-coated sleeve” or the “niobated sleeve”) can be used for joining two components. The sleeve is positioned over the two components to be joined (also referred to herein as the “inner components”), which can in some embodiments be in the form of two tubular components that are substantially longer than the sleeve. The niobium reacts with the NiTi alloy on the outer surface of the sleeve, to produce a eutectic liquid in the manner described in aforementioned U.S. Pat. No. 7,896,222 (the entire contents incorporated herein by reference). The eutectic liquid produced is typically about 3× the volume of the niobium that has reacted, and flows by capillary action on the surface of the sleeve, finding its way into the gap between the overlying sleeve (outer component) and the underlying inner components, i.e., the gap between the outer diameter of the inner components and the inner diameter of the outer component (sleeve). That is, the eutectic liquid flows over the ends (edges) of the sleeve (and or through holes in the sleeve) and through the space (gap) between the inner components and the sleeve, eventually filling this gap. When the assembly is quenched, this effects robust metallurgical attachment of the two components and applies reinforcement of the butt joint of the two components. Various embodiments of the sleeve are disclosed herein.

In some embodiments, the niobium-coated sleeve is configured so it that allows eutectic liquid to flow mainly through intermediate regions of the sleeve, i.e., regions spaced from its ends or edges (some flow can also be over the ends). This flow through the intermediate regions can be achieved by providing the sleeve with micromachined porosity, i.e., fenestrated zones through the wall of the sleeve, spaced from the two ends of the sleeve, as discussed below. In other embodiments, the eutectic liquid is channeled to flow only through these intermediate regions of the sleeve. This is also described in more detail below.

The amount/level of niobium applied to the sleeve needs to be carefully predetermined to effect the desired joining of the components. One volume niobium, as noted above, reacts with approximately two volumes of NiTi alloy, to form a eutectic liquid having a total of three times the niobium volume. Consequently, if there is too much niobium on the sleeve, when heated, it can dissolve the whole structure. That is, an excess of niobium on the surface of the sleeve results in too much eutectic liquid being formed which can cause the destruction of the sleeve, and/or an unwanted erosive attack of the inner components. On the other hand, if there is too small an amount of niobium on the sleeve, an insufficient amount of eutectic liquid is formed that would not adequately fill the critical capillary spaces, such that a weak joint between the components would be formed. Therefore, the amount of niobium needs to be carefully optimized so the proper ratio of niobium coating to the sleeve, i.e., the PVD film or coating thickness on the sleeve surface, is applied.

For this ratio, let the optimum niobium coating thickness on the outer diameter of the sleeve be given as a fraction of the sleeve wall thickness, which is directly proportional to the volume ratio of coating to sleeve. Then if this ratio is greater than one half, the eutectic reaction would consume the whole of the sleeve, an undesired result. Therefore, in preferred embodiments, the desired ratio of coating thickness to sleeve wall thickness would be ≤½.

Additionally, the absolute thickness of the niobium coating or film that is required on the sleeve is related to the size of the gap between the sleeve and the inner component(s) to be joined, the gap defined as the space between the inner diameter of the sleeve and the outer diameter of the inner components to be joined (and referred to herein as the “sleeve-to-inner-component gap”). This gap is proportional to the amount of eutectic liquid needed to fill it. The volume of this gap may be taken as an absolute minimum volume of eutectic liquid needed, which should be corrected upwards for diversion of eutectic liquid to capillary sinks outside the gap. Therefore, if a gap of a given thickness is to be filled, in preferred embodiments, then a niobium coating of one half this thickness would provide enough eutectic liquid to fill this gap, with 50 percent to spare.

Consequently, the preferred niobium coating thickness on the outer diameter of the sleeve is between one half the sleeve wall thickness, at a maximum, and, at a minimum, one half the thickness of the sleeve-to-inner-component gap.

A refinement of the foregoing optimized ratios discussed above can be made taking into account how much unreacted nitinol sleeve is desired to remain, unreacted, at the joint, to function as a reinforcement. (After the brazing process, the now-brazed sleeve may be subjected to post-braze centerless grinding operations and other finishing protocols such as electropolishing).

In preferred embodiments, at minimum, the maximum thickness of the Nb layer is less than or equal to ½ the thickness of the wall of the sleeve. In more preferred embodiments, the maximum thickness of the Nb layer is less than or equal to ¼ the thickness of the wall of the sleeve Note in such embodiments, portions of the sleeve will be left behind and not dissolved into the eutectic liquid, creating a reinforcement. In other embodiments, for example, the niobium coating thickness is between about 1% and about 15% of the sleeve wall thickness, and in more particular embodiments between about 2% and about 10%, and in more particular embodiments between about 1.8% and about 9.2% of the sleeve wall thickness and in other more particular embodiments between about 2.9% to about 14.6%. (Note “about” can mean for example a range of ±10% or ±20% of the given percentage). Note the thickness of the niobium coating can be higher when joining solid components than when joining tube components since too much niobium could eat through the walls of the tubes due to their thinner walls. In some embodiments, the niobium by way of example has a thickness between 1 to 5 microns.

The required niobium coating thickness does not depend on the length of the sleeve because all of the volumes discussed above vary directly with this length, such that the ratios mentioned are not changed if the length of the sleeve is changed. However, the preferred length of the sleeve, in order for it to have ease of handling, and so that it acts as adequate reinforcement of the joint, is preferably ten times the outer diameter of the inner components, e.g., hypotubes, to be joined, but may for example be as little as five times, or for example as great as one hundred times this diameter. In any case, preferred sleeves for joining hypotube-gauge components will be only a few millimeters in length, and many individual sleeves may be mass produced from off-the-shelf nitinol hypotube stock. In the present invention, the niobium required to create a sufficient liquid volume to fill critical capillary spaces is applied to the outer surface of the sleeve material, in a batch process, in the knowledge that the liquid formed during the eutectic reaction (contact melting) will flow along surfaces and collect in the capillary spaces between the sleeve and tubes that constitute the reinforced metallurgical joint.

Referring now in detail to the drawings wherein like reference numerals identify similar or like components throughout the several views, the sleeves in preferred embodiments are manufactured from a plurality of long superelastic NiTi tubes having an inner diameter selected so that a close fit, e.g., a friction-fit, can be made between the sleeve and the inner components (e.g., tubes) to be joined. Preferably the inner diameter of the sleeve is 5-12 micrometers greater than the outer diameter of the inner components (e.g., tubes) to be joined, although other dimensions are also contemplated. A plurality of these tubes, each long enough to be later cut into multiple individual laser-cut sleeves, are preferably niobium coated in a single deposition batch before being cut into separate tubes (sleeves).

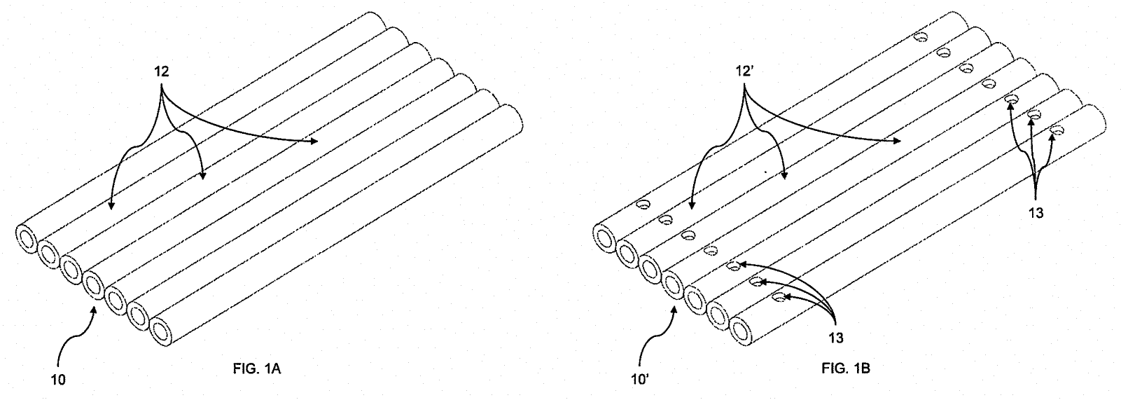

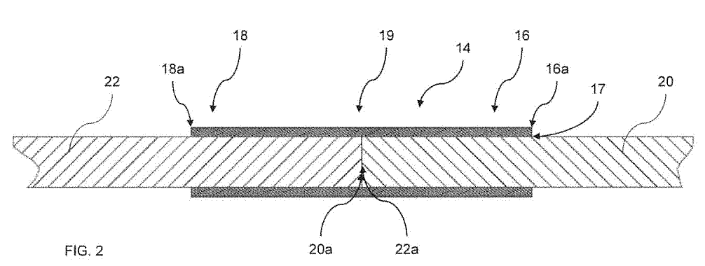

The tubes 12 may positioned side by side, e.g., their longitudinal axes are parallel, and attached to adjacent tubes forming a row of parallel tubes 12. These tubes 12 could then be coated by sputter deposition of pure niobium on both sides. Other deposition methods can alternatively be utilized. Various arrangements of the plurality of tubes are contemplated. For example, the tubes 12 could be fixed in a line (parallel) as shown in FIG. 1 or in a cylindrical arrangement for processing in circular magnetron sputtering devices. The sets of tubes could include a large number of tubes, e.g., 50-100, however, clearly, batches of a different number of tubes are also contemplated. A batch of 100 tubes each 100 millimeters long for example could yield well over a thousand finished sleeves. The niobium can be deposited to the uncut tubes in bulk, before they are laser machined to provide fenestrations or other features and separated into the individual sleeves or alternatively niobium can be deposited to the uncut tubes in bulk, but after they are laser machined to provide fenestrations or other features and then after application of niobium separated into the individual sleeves. Thus, tubes 12 can each be of the desired length and separated into tubes of that length. The tubes can alternatively be of a greater length so that the batch tubes are also cut transversely to provide a number of tubes from each elongated tube of the batch (set).

The advantage of the batches is that multiple tubes can be niobium coated from the stock. This reduces the manufacturing costs since multiple tubes can be formed at one time. Additionally, since the tubes are initially part of an attached set, it facilitates forming of the tubes since it avoids the difficulties involved with handling of the small tubes if they were individual units. Once niobated (or before niobated in some embodiments), the tubes can be laser cut to provide features such as slots, holes, etc., as described herein. In preferred embodiments, only after these laser cut modifications are complete are the niobated tubes finally laser cut into individual tubes for use as the individual sleeves described herein. The final dicing can in some embodiments occur at the time of mechanical assembly of the joint.

It should be appreciated that although not preferred, in some embodiments of the present invention, the sleeves can be niobated after cut into individual tubes.

The tubes of the set can be of desired length for intended applications and in some embodiments by way of example are each about 3 inches to about 4 inches, although other lengths are contemplated. (As noted above, the tubes can also be cut to this desired length from a longer tube). In the embodiment of FIG. 1A, the tube stock 10′ is laser cut to provide holes 13 prior to separation of the tubes 12′ from the stock 10′ as discussed below. (The function of the holes (fenestrations) 13 for liquid flow is discussed below). Otherwise, the batch 10′ is the same as batch 10 of FIG. 1. The tubes can have an outer diameter of about 0.020 inches for example, although other diameters are also contemplated.

In one embodiment by way of example, sleeves meant for joining two hypodermic-scale tubular components that are each of a length of several tens of centimeters and a diameter of about 0.4 millimeters, are made from a batch of tubes 12 which are small superelastic NiTi tubes and each have a length of about 4 inches and a diameter of about 0.020 inches, with inside diameters as described herein. The tubes can then be separated, i.e., laser cut, from the batch after application of niobium if a solid tube is desired, or alternatively, separated after application of niobium and laser micromachined to add features such as fenestrations, slots, etc. Note tubes of different lengths and diameters (which form the outer sleeve) are also contemplated, depending on the components to be joined.

FIG. 2 illustrates in accordance with one embodiment, a niobium coated sleeve 14 placed over two metal tubes 20, 22 to be joined, these tubes being different NiTi alloys, or a NiTi alloy and another biomedical metal such as 316 or 304 stainless steel (which can be coated for example with platinum or tantalum), platinum or tantalum or alloys of the foregoing. The niobium-coated sleeve 14 is made from one of the tubes 12 of set 10 of FIG. 1. As shown, sleeve 14 has a first end portion 16 terminating at a first edge 16a, a second end portion 18 at the opposing end terminating at a second edge 18a, and an intermediate portion 19 between end portions 16 and 18. The sleeve 14 has a solid surface along its length forming an open cylinder. Tube 20 has a first end 20a and a second opposing end, tube 20 extending past edge 16a of sleeve 14 so it is not covered by sleeve 14. Tube 22 has a first end 22a and a second opposing end, tube 22 extending past edge 18a so it has a region not covered by sleeve 14. Ends 20a and 22a are placed in abutment for formation of a butt joint. This joint is reinforced by the sleeve 14 which overlies the abutting end regions. Gap 17 is formed between the outer diameter of the inner tubes 20, 22 and the inner diameter of the sleeve 14. This gap 17 is filled with eutectic liquid formed on the surface of the niobium-coated sleeve when the liquid migrates over the surface of the sleeve and is drawn into the gap 17 by capillary forces in accordance with the heating process described herein.

Various alternate embodiments of the niobium-coated sleeve will now be discussed. In the embodiment of FIG. 4, the sleeve 30 with niobium coating 31 has a solid surface along an intermediate portion between slotted ends forming fingers 32, 34. In this embodiment, the eutectic liquid will flow around the slots and the opposing first and second edges and into the gap between the inner diameter of the sleeve 30 and the outer diameter of the underlying components (e.g., tubes). When heated, eutectic liquid does not enter through the intermediate regions or outer surface (wall) of the sleeve 30 as the outer surface (wall) is continuous (solid) along its length.

In an alternate embodiment, the niobium-coated sleeve has micro fenestrations for entry of eutectic liquid through intermediate portions of the outer surface (wall) of the sleeve. As shown in the embodiment of FIG. 3A, sleeve 40 has series of micro fenestrations 42 (only some are labeled for clarity) along its outer wall 43 spaced about the circumference between opposing edges 44 and 46, passing through the entire thickness 47 of the wall to communicate with the internal lumen 48 of sleeve 40. The fenestrations (holes) 42 are preferably formed by laser cutting and formed prior to separation of the tubes from the bulk sheets 10 (10′) of stock material. A various number of fenestrations can be provided and the number and pattern of fenestrations in FIG. 3A is one example of a fenestrated sleeve. In use, when heated, the fenestrated sleeve 40 promotes rapid infusion of eutectic liquid from the surface of the sleeve 40 to the gap (space) between the sleeve 40 and underlying components (e.g., two tubes) through the fenestrations in addition to the infusion that may occur around the edges 44, 46 of the sleeve 40.

In one example, a fenestrated sleeve having a length between about 3 mm to about 5 mm is placed over two different NiTi alloy tubes to be joined. The sleeve has an outer diameter of 0.0185 inches and an inner diameter of 0.0145 inches. The wall thickness is therefore 0.002 inches. The fenestrations extend though the entire wall thickness and are of a diameter of between 0.0005-0.001 inches. When heated the eutectic liquid flows through the fenestrations as well as around the ends of the sleeve.

FIG. 3B illustrates an alternate embodiment of a fenestrated sleeve, designated by reference numeral 50. In this embodiment, the fenestrations 52 of sleeve 50 are placed along the perimeter (circumference) of the sleeve 50 in the regions of the sleeve 50 near the quarter points of its length. This advantageously places the fenestrations (holes) 52 within the early melt pool during laser heating. Consequently, they will more reliably carry eutectic liquid to the underlying tube/sleeve interface. Thus, in this embodiment, the end of the sleeve 50 would not be needed as a eutectic liquid infiltration route. In other words, in this embodiment, eutectic liquid would be drawn to the zone in the joint where it is most needed—at the butt-gap 55 in the center of the sleeve. Note in this embodiment, there are no fenestrations directly over the butt so that a hermetic seal is formed where the inner components meet.

Although vacuum furnace heating can be used to initiate the eutectic melting event, preferably laser irradiation is utilized to selectively heat the sleeve without excessively heating the inner objects (components) to be joined. The embodiment shown in FIG. 3B in certain applications can avoid overheating of the underlying tubular components. That is, in irradiating the sleeve with a laser, such as a galvo scanning laser, it is desirable not to over scan the sleeve, e.g., not to apply the laser energy past the ends of the sleeve. This is to avoid overheating the inner components where they emerge from the overlying sleeve. However, the ends of the sleeve have a greater radiating surface area (because of the end faces), and also suffer a conduction heat-loss to the inner components. Thus, the ends of the sleeve will require more laser power input to reach a given process temperature. Although this extra local energy input can be arranged using galvo scanning software, doing so carries the risk of overheating the emerging tubular components. In any reasonable laser irradiation protocol, the two sleeve ends can be expected to be cooler than the center. There is likely to be a central pool of first-melted eutectic liquid that may reach the ends of the sleeve. If this were the case, there would be a longer infiltration route for the eutectic liquid to get down into the tube/sleeve gap, which needs to be filled in order that the joining of the inner components is effected. With such a central pool, and without fenestrations, the eutectic liquid might remain trapped in the middle, or drip off, resulting in a bad joint. The use of the fenestrations 52 of FIG. 3B addresses this dilemma by their placement on the sleeve 50 and their communication with the inner lumen 51 of sleeve 50. As shown, the fenestrations 52 are spaced from the center point 54 (which overlies the abutting ends of the underlying tubes 53, 57 shown in phantom) and spaced from the ends 54, 56 of sleeve 50. The dashed radially extending lines 58, 59 at the end regions of sleeve 50 illustrate schematically the approximate region of eutectic liquid flow, with the eutectic liquid penetrating the sleeve 50 and not reaching the ends of the sleeve outside the dashed lines 58, 59. (Outside defined as to the right and left of the lines 58, 59 respectively, as viewed in FIG. 3B) Thus, a limited melt or eutectic liquid reaction zone is provided and the ends of the sleeve 50 stays relatively cool.

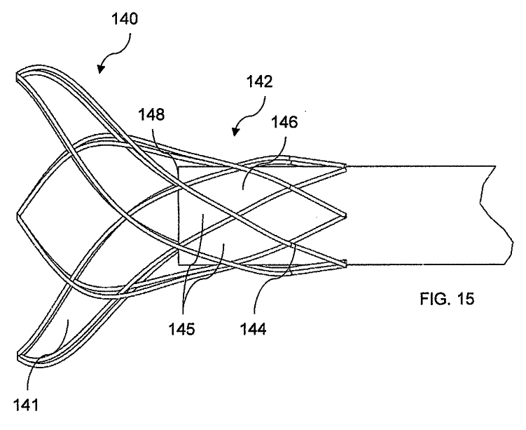

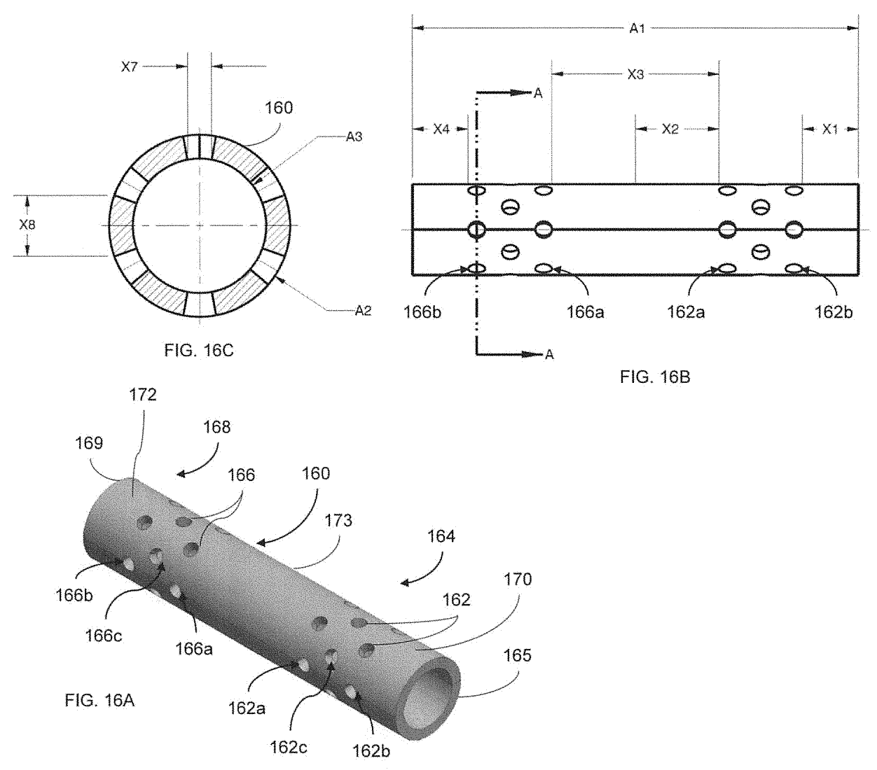

FIGS. 16A-16C illustrate an alternate embodiment of a fenestrated sleeve (outer component). Sleeve 160 has a first set of fenestrations (openings) 162 at end region 164 spaced from first edge 165 and a second set of fenestrations 166 at opposing end region 168 spaced from second edge 169. First set of fenestrations 162 has an inner array 162a, and outer array 162b and a middle array 162c between arrays 162a, 162b. Similarly, second set of fenestrations 166 has an inner array 166a, and outer array 166b and a middle array 166c between arrays 162a, 166b. The outer arrays 162b 166b are spaced from the respective first and second edges 165, 169 leaving a solid surface region 170 between outer array 162b and edge 165 and a solid surface region 172 between outer array 166b and edge 170. Note each of the arrays includes a series of spaced apart holes extending circumferentially around the sleeve in a 360 degree arc, i.e. in a ring like manner around the sleeve. Also, note in the illustrated embodiments the middle arrays 162c, 166c are staggered with respect to their inner and outer arrays. Intermediate region 173 between the two sets of fenestrations 164, 166 is a solid surface without fenestrations. Note in this embodiment, each of the 6 arrays has 6 holes for a total of 36 holes in the sleeve, although a fewer or greater number of arrays and holes are also contemplated.

Below are charts showing two examples of dimensions of the sleeve 160, with the dimensions/areas demarcated in FIGS. 16B and 16C. However, it should be appreciated that these dimensions are provided by way of example as other dimensions and arrangements of openings are contemplated. It should also be appreciated that a different number of fenestrations, a different number of arrays of fenestrations and different arrangement, e.g., all arrays staggered, non-staggered, etc. are also contemplated.

### Example 1

| | | Measurement |
|---|---|---|
| Designation | Description | (inches) |
| A1 | Length of sleeve (distance between edges) | .080 + .005 |
| A2 | Outer diameter of sleeve | .0165 |
| A3 | Inner diameter of sleeve | .0138 |
| X1 | Distance from first edge to outer array | .010 |
| | of openings of first set | |
| X2 | Distance from inner array of openings of | .015 |
| | first set to center point | |
| X3 | Distance between inner array of openings | .030 |
| | of first set and inner array of openings | |
| | of second set | |
| X4 | Distance from edge to outer array of | .010 |
| | openings of second set | |
| X5 | Distance from inner array of openings | .015 |
| | of second set to center point | |
| X6 | Distance from outer array of openings | .015 |
| | of second set to inner array of openings | |
| | of second set | |
| X7 | Width of opening | .0025 |
| X8 | Spacing between adjacent holes along arc | .0055 |
| | of sleeve | |

### Example 2

In Example 2, all dimensions are the same as Example 1 except the following

| | |
|---|---|
| Inner diameter of sleeve | .0122 |
| Radial hole width | .0022 |
| Radial hole spacing (Spacing between adjacent | .0055 |
| holes along arc of sleeve) | |

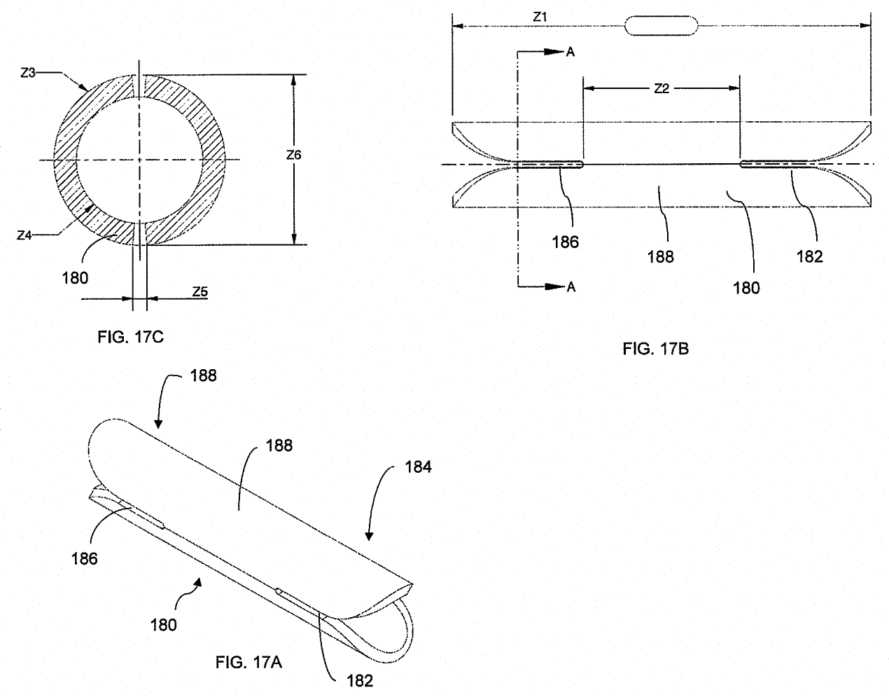

FIGS. 17A and 17B illustrate an alternate embodiment of a sleeve (outer component) 180 having slots instead of fenestrations. Sleeve 180 has slots (openings) 182 at first end 184 and slots 186 at second end 188, leaving solid region 188 between the slots 182, 186. The slots 182, 186 facilitate insertion of the inner component into the sleeve 180. The slotted ends also reduce the stiffness of the sleeve 180 and provide increased compliance at the ends. Below is a chart showing an example of dimensions of the sleeve 160, with the dimensions/areas demarcated in FIGS. 17B and 17C. However, it should be appreciated that these dimensions are provided by way of example as other dimensions and arrangements of openings are contemplated. Additionally, a different number of slots could be provided.

### Example 3

| | | Measurement |
|---|---|---|
| Designation | Description | (inches) |
| Z1 | Length of sleeve (distance between edges) | .080 + .005 |
| Z2 | Distance between slots | .030 |
| Z3 | Outer diameter of sleeve | .0165 |
| Z4 | Inner diameter of sleeve | .0138 |
| Z5 | Slot spacing on ID | .0010 |

In an alternate embodiment (Example 4), the dimensions can be the same as Example 3 except the ID of the sleeve can be 0.0122 inches.

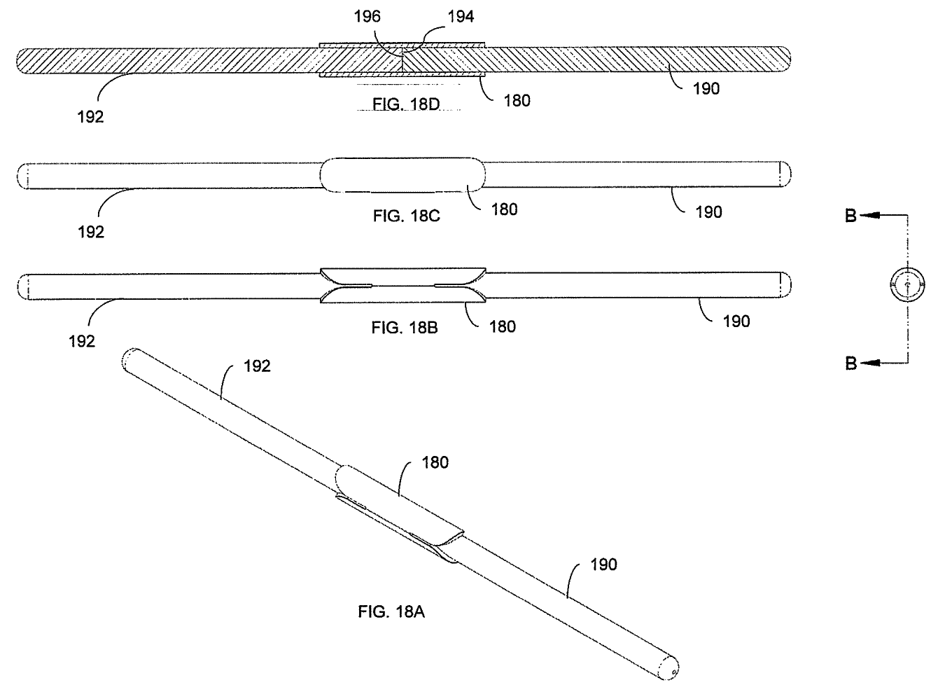

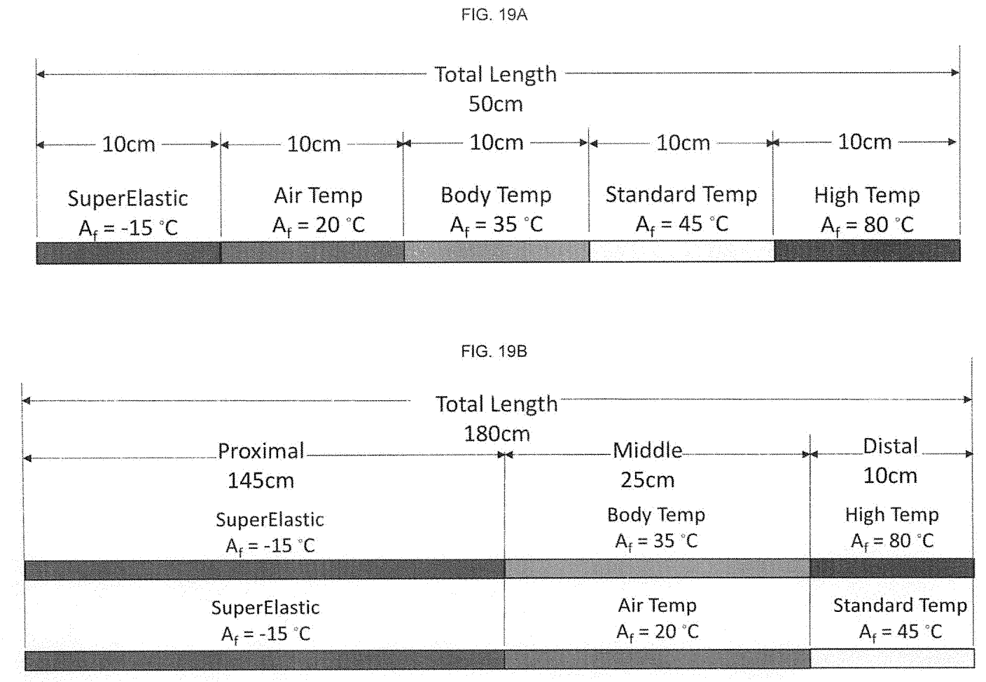

FIGS. 18A-18B illustrate use of the slotted sleeve 180 of FIG. 17A to join two components to form a device such as a guidewire. Components 190 and 192 are inserted into the opposing ends of the sleeve 180 and placed so inner edges 194, 196 of components 190, 192, respectively, are in abutment to be joined by the Niobium sleeve in accordance with the methods described herein. Note the inner components 190, 192 underlying the sleeve 180 are shown as solid wires or tubes, however, as noted herein, the inner components could alternatively be hollow tubes.

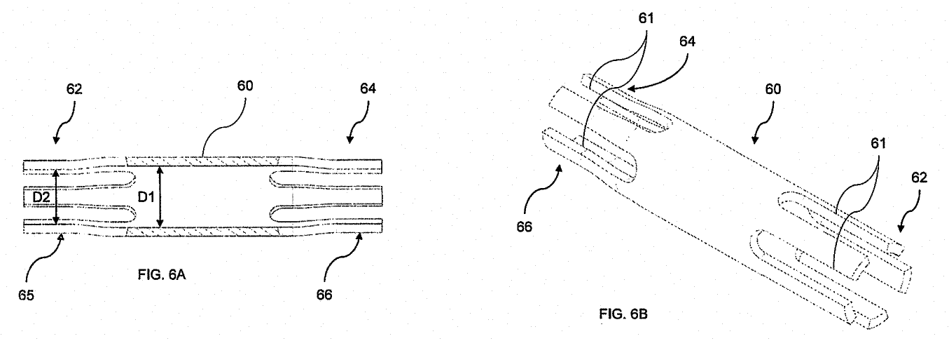

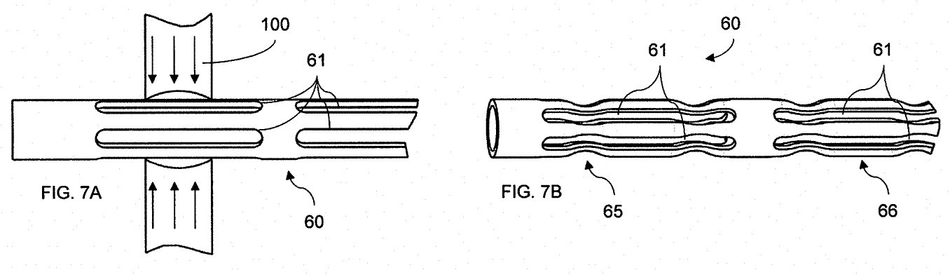

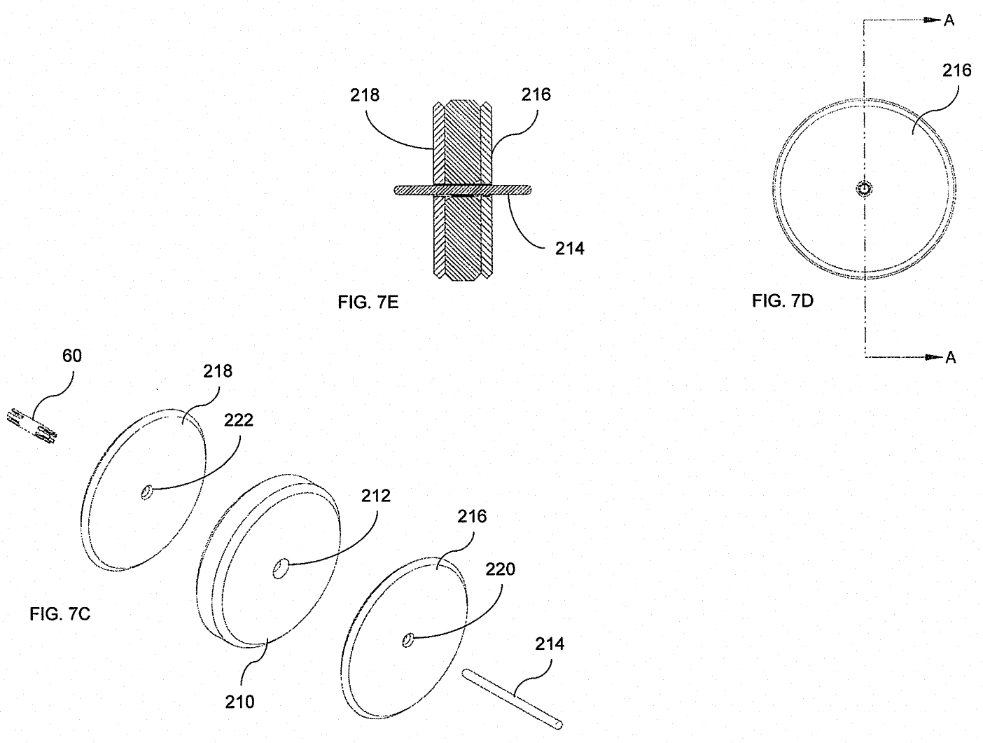

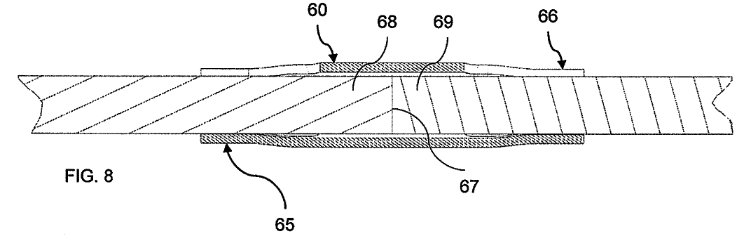

In the foregoing embodiments, the sleeves are placed over the components, e.g., the two inner (e.g., tubular) components to be joined, and initially held by a friction fit. In alternate embodiments, the sleeve is provided with a gripping or retention feature to enhance retention of the internal components during processing. Such gripping/retention feature can be utilized with any of the sleeves disclosed herein, e.g., the solid sleeve, fenestrated sleeve, slotted sleeve etc. This provides for additional gripping of the inner wires or tubes, e.g., hypotubes, for ease of handling at the time of assembly/manufacture. FIGS. 6A and 6B illustrate an example of a sleeve having a retention feature. Sleeve 60 has a series of axially (longitudinally) extending slots 61 formed at first end 62 and second opposite end 64 forming spaced apart fingers. Ends 62 and 64 are hot shaped swaged to form a reduced diameter region 65, 66 at each end. These reduced diameter regions increase the retention force on the inner component during assembly as it elastically grips the inner component to provide a tighter fit, e.g., enhances the friction fit between the outer sleeve and inner components. Thus, the resulting sleeve has a slotted/solid/slotted configuration, with the solid being in the intermediate portion between the two slotted portions. As shown, it is crimped down at the edge but then flares up (outwardly) at the end. In some embodiments, the crimp can reduce the inner diameter D2 to about 10% to about 20% of the inner diameter D1 of the sleeve 60. The radial flare in a direction away from the longitudinal axis facilitates insertion of the inner components through the ends of the sleeve. Note to aid understanding of the process, FIG. 6B shows both ends slotted but only one end swaged. FIGS. 7A and 7B show one method of forming the swaged ends, the swaged ends preferably formed on the tube by tool 100 while attached in the bulk set of FIG. 1A (or FIG. 1B). In some embodiments, the slots are formed on the tube stock spaced from the ends of the tubes and then the end material is removed so the slots are at the very ends of the sleeve; in other embodiments, the slots are formed on the very ends of the tube stock so the end material does not need to be removed for forming the sleeve. FIG. 8 shows sleeve 60 over tubes 68, 69 to be joined at butt joint 67. Sleeve 60 can also have fenestrations as in the embodiments of FIGS. 3A, 3B and 16A. FIGS. 7C-7E show an alternative method for forming the swaged ends. Support disk 210 has a hole 212 to receive the sleeve 60. A wire 214 extends through the sleeve 60 to keep the ends of the fingers of the sleeve 60 open when swaged. After the sleeve is centered within the support disk 210, disks 216 and 218, having smaller respective holes 220, 222 are forced over the fingers of sleeve 60 to swage or shape set the fingers (slotted ends). Note this swaging process can be performed when it is cold.

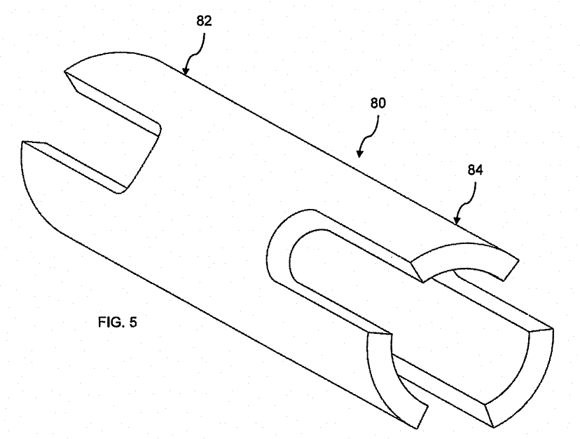

In the alternate embodiment of FIG. 5, the ends of sleeve 80 are slotted as in the ends of FIG. 6A but not swaged. The slotted ends 82, 84 reduce the stiffness of the sleeve 80. This provides increased compliance at the ends. Additional slots or scallops can be provided to reduce the stiffness. A reduction in the stiffness of the sleeve at its ends reduces stress concentrations at this mechanical discontinuity, making the joint more robust. Fenestrations as in FIGS. 3A, 3B and 16A can be provided. The slots, as in FIG. 6A, and the fenestrations, if provided, are preferably formed on the tube stock prior to separation of the individual tubes. Note the slots are along the longitudinal axis in FIG. 5; in FIG. 4 the slots are angled forming different shaped fingers.

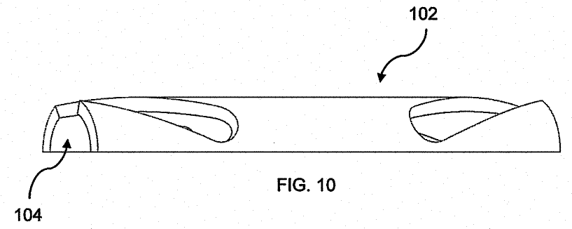

In FIGS. 2-8, the sleeves are cylindrical, having a transverse circular cross-section. In alternate embodiments, the sleeve does not extend around a full 360 degrees. For example, in FIG. 10, sleeve 102 is C-shaped (U-shaped) in transverse cross-section. The sleeve extends around 180 degrees of the inner components, e.g., tubes, to be joined. As can be appreciated, the sleeve can have a greater or lesser “C” so it can extend for more than 180 degrees or for less than 180 degrees. Sleeve 102 has a lumen 104 to receive the two inner components (e.g., NiTi alloy inner tubes). In all other respects, sleeve 102 is the same as sleeve 30 of FIG. 4 and the method for heating the sleeve for eutectic liquid flow as described herein is effected for creating the joint. Note sleeve 102 could be solid or could also have fenestrations, different formed slotted ends, swaged ends, or other features of the other embodiments described herein and the details and function of such features are fully applicable to sleeve 102. Further, the sleeve 102 can be formed from laser cutting a bulk set as in the sleeves 12 of FIG. 1. The inner components to be joined by sleeve 102 can be cylindrical wherein the joint would be formed about less than 360 degrees (e.g., 180 degrees) which would leave a flexible region at the joint. In this manner, the components can flex or bend with respect to each other while still having a strong joint to prevent breaking/separation. That is, one region of the formed tube can move relative to the other region. In other embodiments, sleeve 102 can be used to join inner components being C-shaped in transverse cross-section or of a transverse cross-section matching (or substantially matching) the transverse cross-section of the sleeve. In such embodiments, the C-shaped sleeve could fully encompass the C-shaped inner components to provide a rigid joint by heating as described herein.

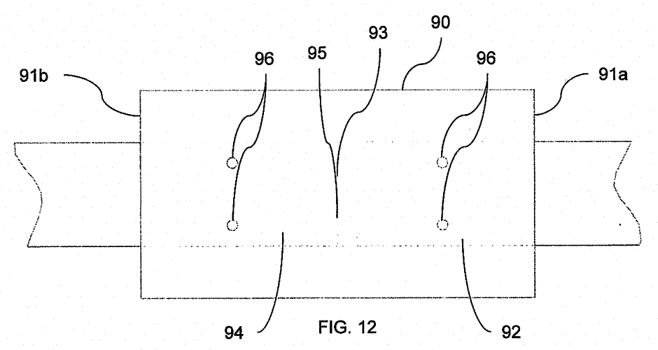

In the foregoing embodiments, circular (or oval) or semi-circular (or semi-oval) sleeves are described to join circular (or oval) or semi-circular (or semi-oval) components. For such joining, circular (or oval) or semi-circular (or semi-oval) sleeves are placed over the components to be joined, preferably fully, but at least partially or substantially, surrounding the outer surface adjacent the abutting end. FIG. 12 illustrates an alternate embodiment wherein instead of a sleeve (tubular or collar component), a planar component (member), such as a flat sheet, is utilized to join two planar components, e.g., two flat sheets of different NiTi alloys (or sheet of Nitinol alloy to metal or metal alloys as described herein) in an end to end fashion. More specifically, niobated sheet 90 is placed over sheets 92 and 94, which are aligned in end to end fashion so edge 93 of sheet 92 abuts edge 95 of sheet 94. The sheet 90 has fenestrations (openings) 96, preferably formed from laser cutting as in the foregoing embodiments, for the flow (infiltration route) of eutectic liquid as in the fenestrations of the foregoing embodiments. Two fenestrations 96 are shown at each end, spaced from ends 91a, 91b of the sheet 90 and spaced from the abutment (butt joint) of sheets 92, 94 for directed flow of eutectic liquid as described above with respect to the embodiment of FIG. 3B. In alternate embodiments, a different number of fenestrations could be provided, and at different locations to provide the eutectic liquid infiltration route. The planar member 90, which has been coated with niobium by one of the aforementioned deposition techniques, is placed over the two sheets 92, 94, with portions of sheets 92, 94 extending beyond (exposed from) ends 91a, 91b of planar member 90, and melting causes the eutectic liquid to flow through openings 96 to form the reinforced metallurgical joint of sheets 92, 94 via the process described herein. In alternative embodiments, the planar member can have openings along its length as in the openings of FIG. 3A to provide additional openings at the intermediate region for eutectic liquid flow. The sheet 90 can also be designed so that eutectic liquid flow is around edges 91a, 91b into a gap between sheet 90 and the underlying sheets 92, 94. Such flow around the edges 91a, 91b can be in addition to the flow through the fenestrations, or if fenestrations are not provided, flow around the edges can provide the sole route of fluid flow to effect joining of the components 92 and 94. The individual sheets for effecting joining can be formed from a large sheet of material, which is coated with niobium and fenestrations laser cut (either before or after coated) prior to the individual sheets being separated (cut) from the large sheet of material. This enables multiple sheets to be formed from a single sheet providing the manufacturing and handling advantages described above with respect to the bulk set of tubes of FIG. 1 (and FIG. 1A). Alternatively, the sheets can be coated with niobium and/or fenestrated after separation from the large sheet.

It is also contemplated that the planar component 90 can be used to join components that are not planar, e.g., are tubular or have curved surfaces. This would provide a flexible joint since the components would not be joined around the full circumference.

The foregoing embodiments illustrate use of the niobium-coated sleeve to attach two components end-to-end, i.e., forming a butt joint. However, the niobated sleeve concept disclosed herein can also be utilized in alternate embodiments to attach an outer component over a single inner component. For example, as illustrated in FIG. 9, the sleeve 70, also referred to as collar, is positioned coaxially (concentrically) over the inner component 72, with both ends 75, 76 of the inner component 72 extending outside the sleeve 70. The sleeve 70 has a niobium coating or film thereon as described above. When laser heated, the eutectic fluid flows around the ends of the sleeve 70 into the space 74 between the inner diameter of the sleeve 70 and the outer diameter of the inner component 72, thus joining the two components 70, 72. The sleeve 70 can have a friction fit or can have swaged ends or other retention features to enhance gripping the inner component 72. The sleeve 70 can also have fenestrations as in the embodiments of FIG. 3A or 3B for flow of eutectic liquid through the outer surface (wall) of the sleeve. The sleeve 70 could also have slots as in the slots of FIG. 5, 6B or 17A. In one example, the niobated collar is slid over a Nitinol wire having an outer diameter of 0.005 inches and is heated forming a eutectic liquid to melt and bond the collar onto the wire. This joins the outer member (sleeve) to the inner member. Such applications could include for example anti-migration features on stents, features on delivery systems that allow devices to be deployed, etc.

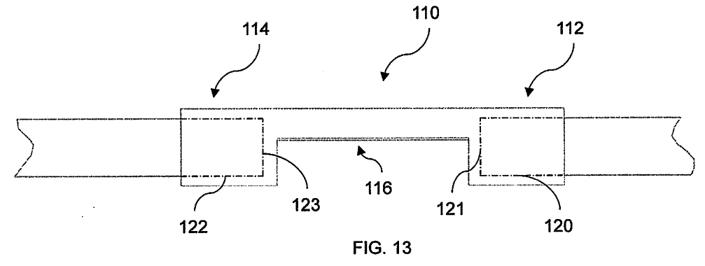

In the embodiments described above, the components are joined end to end, forming a rigid joint between the two components so the two components are fixed with respect to each other. In the embodiment of FIG. 13, the two components are joined in an axially spaced fashion such that the niobium coated sleeve bridges the two components. In other words, in this embodiment, the niobium coated sleeve forms a connector or bridge for the two inner NiTi alloy components to provide a flexible joint. FIG. 13 illustrates one such niobated sleeve, designated by reference numeral 110. Sleeve 110 has a niobium coating at least at its ends 112, 114 for joining two inner components 120 and 122. The two inner components 120, 122 are axially (longitudinally) aligned but axially spaced so inside edge 121 of component 120 is spaced from inside edge 123 of component 122. As shown, reduced diameter region 116 of sleeve 110 extends between the two ends 112, 114. The reduced diameter 116 can be radiused or can have a flattened surface. If flattened, e.g., shape set flat, preferably there are rounded transitions to the ends 112, 114 to prevent fracturing of the sleeve 110. The reduced diameter middle (intermediate) section 116 can in certain instances impart spring like characteristics. The sleeve 110 has a niobium coating at its ends, coated utilizing any of the various deposition processes described herein, and when heated the eutectic liquid flows around the outer edges of ends 112, 114 into the spaces between the inner diameter of the ends 112, 114, and the respective inner components 120, 122 (as in the manner of sleeve 12 of FIG. 2) and/or flows through fenestrations (if provided) in ends 112, 114 (in the manner for example of sleeves 40, 50 or 160 of FIG. 3A, 3B or 16A) or around ends and through slots (in the manner for example of sleeve 30, 60 or 180 of FIG. 4, 6A or 17A). The resulting structure enables flexing of components 120 and 122 with respect to each other. Since the inner components 120, 122 are spaced, different sized (diameter) inner components 120, 122 can be joined providing a final product of varied diameter. The length and/or height (thickness) of middle section 116 of sleeve 110 can be varied to vary the flexibility of the joint.

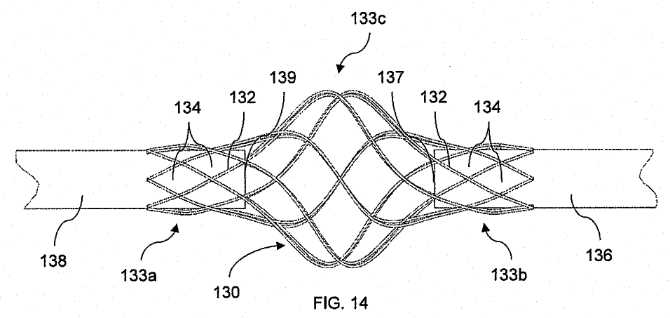

In the embodiment of FIG. 14, niobated sleeve 130 is a braid structure, formed by wound or woven shape memory elements 132 with spaces (also referred to as gaps or openings) 134 between the elements 132 to enable flow of eutectic liquid therethrough. The spaces 134 provide an alternative to the openings (fenestrations) in the embodiments of FIGS. 3A and 3B. The spaces 134 can be varied by changing the tightness of the braid 130 along the length of the braid. The spaces 134 can also vary in different regions of the braid by varying the braid tightness (cell structure) at select regions. For example, larger spaces can be provided at the end regions 133a, 133b to provide openings as described above in the embodiment in FIG. 3B to enable liquid flow and intermediate region 133c can have smaller spaces or the braid can be sufficiently tight at the intermediate regions to effectively preclude eutectic liquid flow in the region which is not overlying the inner components. The braid 130 is shown positioned over inner components, e.g., NiTi alloy tubes 136, 138, to be joined. The two inner components 136, 138 are axially aligned but axially spaced so inside edge 137 of component 136 is spaced from inside edge 139 of component 138. Thus, the braid 130 forms a bridge or connector to provide flexibility between the components 136, 138 so that the components 136, 138 are movable with respect to each other, movement including flexing, bending, etc. The braid 130 can also have an enlarged diameter region 135 between the two joined components which can be useful in certain applications.

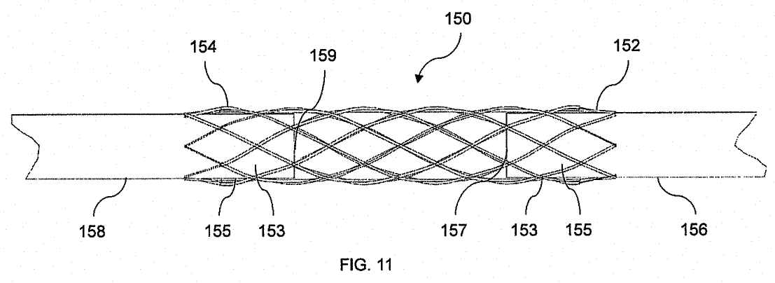

In the embodiment of FIG. 11, braid 150 does not have an enlarged region but has a substantially uniform diameter along its length. In all other respects, braid 150 is identical to braid 130, with niobium coated ends 152, 154 overlying axially spaced longitudinally aligned inner components 156, 158, respectively. As shown, inner (inside) edge 157 of component 156 is axially spaced from inner (inside) edge 159 of component 158). As in braid 130, the cell structure of braid 140 can be varied. Eutectic liquid flows through spaces 155 between elements 153 in the regions overlying the inner components, and the cell structure can in some embodiments be tight at the regions not overlying the inner components so that fluid does not flow through at these regions. Such braids can also be used to connect to a single inner component as in the braid 140 of FIG. 15. More specifically, braid 140, which forms the niobated sleeve, is joined to inner component 146 at end 142. End 142 is coated with niobium in the deposition processes described herein and when heated the eutectic liquid flows into the spaces (gaps) 145 between the shape memory elements 144. It is also contemplated, depending on the cell structure/tightness of the braid, that the eutectic liquid can in addition or alternatively flow around the edge 148 of end 142 between the inner diameter of end 142 and the outer diameter of the inner component 146. Such flow around the edges can also occur if desired around the edges of the braid 130 and braid 150 of FIGS. 14 and 11, respectively. Region 141 can in some embodiments have tight cell structure so that eutectic cannot flow through region 141 of braid 140.

The braids of FIGS. 11, 14 and 15 can be formed individually or alternatively can be individually cut from a stock of braids or from a long braid which can be cut into several smaller braids.

With the description above, it can be appreciated how devices of different properties and/or different materials can be joined by the niobated sleeve. FIGS. 19A-19C show an example of application of the method to form a device having varied properties along its length.

FIG. 19A illustrates how components of different properties can be joined together by the methods of the present invention to form a device with different properties along its length. One device with such differing properties can be a guidewire such as disclosed in provisional application 62/791,693, filed Jan. 11, 2019, the entire contents of which are incorporated herein by reference. The chart of FIG. 19A shows an example of components that have different austenitic finish temperatures (A_{f}) to vary the stiffness and flexibility/malleability along its length. The length of the device in this example is 50 cm with the different regions having a length of 10 cm for equal division of the regions. However, it should be understood that different lengths can be provided and the regions can be of different lengths than shown in the chart and the lengths can be equal or unequal. Moreover, five differing regions are shown, however it should be understood that a fewer or greater number of regions with different Af temperatures can be provided, FIG. 19A providing one example. As shown in FIG. 19A, one region, e.g., the proximalmost region has an A_{f}=−15° C. (Centigrade); the adjacent distal region has an A_{f}=20° C. (equal to air temperature); the next distal region has an A_{f}=35° C. (equal to body temperature); the next distal region has A_{f}=45° C. and the distalmost region, which is the most malleable has an A_{f}=80° C. Thus, in this example, the distalmost region provides the most flexibility and is malleable so it can be shaped by the user. The next region is less malleable, the middle region exhibits some flexibility at body temperature when in use and the two proximal regions provide stiffer regions. This could have application to guidewires where the distal end has more flexibility for steering through the vasculature and the proximal end is stiffer for pushability. This is also shown schematically in FIG. 19C wherein the device is shape set straight and as it is warmed, the segments return to their straightened configuration.

FIG. 19B provides a chart illustrating two examples of guidewires having three sections/zones of different properties with the superelastic material with a lower austenitic finish temperature A_{f} at a proximal section to provide stiffness for pushability, a middle section which has some malleability at body temperature and a distal section with an A_{f} of 80 degrees C. being highly malleable so the user can shape the tip. Note at Aₜ the material is austenitic and will behave superelastically. Note below the A_{f} the material moves toward a more malleable condition (toward a martensitic state) is not as stiff and above A_{f} the material is stiff). The temperatures and the lengths of each section are provided by way of example as it can be appreciated that other temperatures and other lengths are also contemplated.

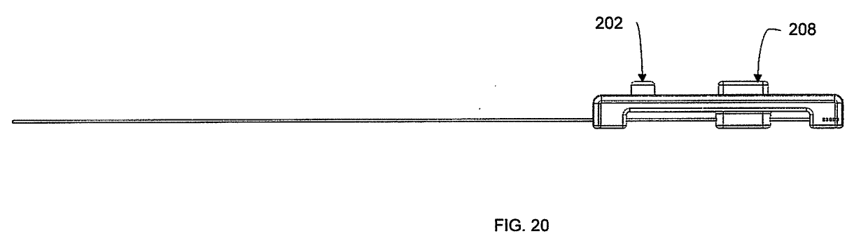

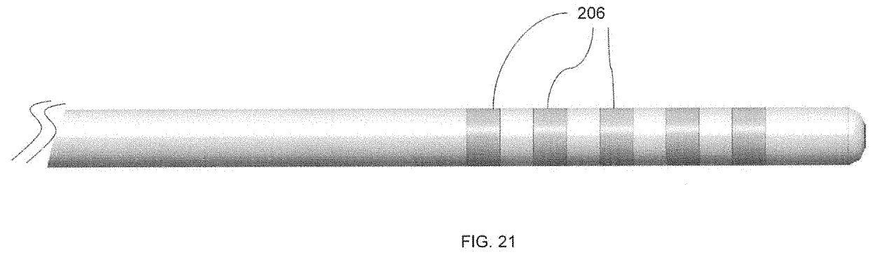

FIGS. 20 and 21 illustrate a system for changing the shape and/or stiffness of the device during the surgical procedure by heating select portions of the device. That is, heat can selectively be applied to the device to increase the temperature to decrease the stiffness of the device. Heating elements can be provided external or internal the device at select regions. It can be battery powered and potentially Bluetooth to a phone/tablet for more finite control and feedback.

The system includes a control box having a stiffness changing button 202. Button 202 is operable to increase the stiffness of the device by applying heat to selected regions of the device. Wires extend from the box through the device into contact with the electrical contacts (heating elements, e.g., heating coils) 206 on the device to heat select contacts and regions. The number of contacts can vary and preferably insulation is provided between contacts. A wire lock button 208 is actuable to clamp the wire within the box 200 to maintain the wire on position. The wire lock button can in some embodiments be spring loaded and in a normally clamped (closed) position wherein it is released for wire insertion. The various sections/regions of the device can be electrically heated during insertion to adjust the stiffness during insertion or during use. After insertion, the device can be detached from the control box 200. Note cold fluid can be injected to cool regions of the device to reduce the stiffness of the desired regions.

As can be appreciated, use of the sleeve (collar) with a niobium coating or film creates a reinforced butt joint for two axially positioned (end to end) components and a reinforced joint for two coaxially positioned components (joining an outer component to an inner component). The sleeves with a niobium coating or film can also join two axially spaced components to create a connector or bridge between the two components. The components to be joined can be of different lengths. Configurations of the sleeves, e.g., laser cut holes, enable control/direction of eutectic liquid flow to form the reinforced metallurgical joint.

The metallurgical joining of components by the aforedescribed reactive eutectic brazing using niobated sleeves enables joining of superelastic material to superelastic material, shape memory material to shape memory material, shape memory material to superelastic material and stainless steel, tantalum or platinum to superelastic material or to shape material. The attachment to stainless steel for example can provide a super stiff component or alternatively a malleable material, depending on the stainless steel. The joining of components disclosed herein enables not only joining of components of different materials, but components of the same or different material having different properties to form for example a single tube with varying properties along its length, such as a stiffer portion at one end and a more flexible portion at another end, an enhanced radiopaque region at one end, a different diameter at one end, etc. The embodiments wherein the niobated sleeve forms a connector for the two axially spaced longitudinally aligned components provide a device such as an elongated tube with a flexible joint wherein one end of the device is flexible or bendable or otherwise movable with respect to the other end.

The components joined herein can be used for creating medical devices such as guidewires, stents, microcatheters, etc.; however, it also has application outside the medical device area where it is desired to join two components.

The present invention provides a) a method of forming the devices using the niobium sleeve process described herein; b) a device having regions of different properties and a joint formed by a niobium coated nickel titanium alloy sleeve melted thereon; and/or c) a device formed by the process of joining separate components together by a niobium coated nickel-titanium alloy sleeve melted onto the components to form a joint(s).

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure as defined by the claims.

What is claimed is:

1. A method of joining two metal components to form a medical device comprising:
a) positioning a first metal component in a first end of a sleeve, the sleeve composed of a nickel titanium alloy and having niobium deposited thereon;
b) positioning a second metal component in a second end of the sleeve; and
c) increasing the temperature of the sleeve so the niobium reacts and melts to form a joint joining the first and second components.

2. The method of claim 1, wherein the second component is composed of a shape memory or superelastic nickel titanium alloy.

3. The method of claim 2, wherein the first component is composed of platinum or tantalum or stainless steel coated with a metal.

4. The method of claim 1, wherein the sleeve is composed of a shape memory or superelastic nickel titanium alloy.

5. The method of claim 1, wherein the first component has a flexibility less than the second component at room temperature.

6. The method of claim 1, wherein the first component has a flexibility less than the second component at body temperature.

7. The method of claim 1, wherein the first component has an austenitic finish temperature different from the second component.

8. The method of claim 1, wherein the sleeve avoids direct contact with the niobium and the first and second components underlying the sleeve and the niobium for reactive brazing is not applied to the underlying first and second components.

9. The method of claim 1, wherein the sleeve has a plurality of holes in a wall of the sleeve and increasing the temperature of the sleeve causes flow of the melted niobium into contact with a surface of the first and second components underlying the sleeve.

10. The method of claim 9, wherein the plurality of holes are spaced from edges of the sleeve and spaced from the center point of the sleeve.

11. The method of claim 1, wherein the sleeve has a slot at the first and second ends for flow of eutectic liquid into a gap between an inner diameter of the sleeve and an outer diameter of the first and second components.

12. The method of claim 1, wherein a niobium coating thickness on the sleeve is between one half the sleeve wall thickness at maximum and one half thickness of gap between the sleeve and first component.

13. The method of claim 12, wherein the thickness of the niobium layer is between 1% and 15% of the thickness of a wall of the sleeve.

14. A method of forming a joint between a first component composed of nickel titanium alloy and a second component composed of a biocompatible metal or metal alloy, the method comprising placing a niobium coated sleeve over a region between the first and second components and reactively brazing the sleeve to the first and second components to form a brazed joint joining the first and second components.

15. The method of claim 14, further comprising the step of placing the first and second components within opposing ends of the sleeve and in abutment prior to reactive brazing and placing the first and second components.

16. The method of claim 15, wherein during reactive brazing, the niobium flows around edges of the sleeve.

17. The method of claim 15, wherein during reactive brazing, the niobium flows through openings in the sleeve, the openings communicating with an outer surface of the first and second components.

18. A medical device having a first region having a first property, a second region having a second property different than the first property and a joint formed by a niobium coated nickel titanium alloy sleeve melted onto a first section of the first region and a second section of the second region.

19. The medical device of claim 18, wherein the first property is a first stiffness and the second property is a second stiffness greater than the first stiffness.

20. The medical device of claim 18, wherein the first property is a first transition temperature and the second property is a second transition temperature.
